(19) United States
(12) Patent Application Publication
(10) Pub. No.: US 2004/0214203 A1
(43) Pub. Date: Oct. 28, 2004

(54) Genes related to sensitivity and resistance to chemotherapeutic drug treatment

(75) Inventor: John P. Fruehauf, Tustin, CA (US)

Correspondence Address: McDonnell Boehnen Hulbert & Berghoff, 32nd Floor, 300 S.Wacker Drive, Chicago, IL, 60606, US

(73) Assignee: Oncotech, Inc., Tustin, CA (US)

(21) Appl. No.: 10/734,880

(22) Filed: Dec. 12, 2003

Related U.S. Application Data
(60) Provisional application No. 60/432,922, filed on Dec. 12, 2002.

(51) Int. Cl. C12Q 1/68

(52) U.S. Cl. 435/6

(57) ABSTRACT

The invention provides methods for prognosis, diagnosis, staging and disease progression in human cancer patients related to expression levels of a plurality of genes that are differentially expressed in chemotherapeutic drug resistant and drug sensitive tumor cells.

This application claims priority to U.S. provisional patent application Ser. No. 60/432,922, filed Dec. 12, 2002, the disclosure of which is explicitly incorporated herein.

### BACKGROUND OF THE INVENTION

1. Field of the Invention

The invention relates to cancer diagnosis and treatment, and specifically to the determination of a drug resistance phenotype in neoplastic cells from cancer patients. The invention specifically relates to the separation of chemotherapeutic drug resistant neoplastic cells from drug sensitive neoplastic cells and stromal cells. The invention in particular relates to the identification of genes that are differentially expressed in chemotherapeutic drug resistant neoplastic cells compared with the expression of these genes in drug sensitive neoplastic cells. As part of this identification, the invention provides a pattern of expression from a selected number of identified genes, the expression of which is increased or decreased in chemotherapeutic drug resistant neoplastic cells. The invention provides methods for identifying such genes and expression patterns of such genes and using this information to make clinical decisions on cancer treatment, especially chemotherapeutic drug treatment of cancer patients.

2. Summary of the Related Art

Cancer remains one of the leading causes of death in the United States. Clinically, a broad variety of medical approaches, including surgery, radiation therapy and chemotherapeutic drug therapy are currently being used in the treatment of human cancer (see the textbook CANCER: Principles & Practice of Oncology, 2d Edition, De Vita et al., eds., J. B. Lippincott Company, Philadelphia, Pa., 1985). However, it is recognized that such approaches continue to be limited by a fundamental inability to accurately predict the likelihood of clinically successful outcome, particularly with regard to the sensitivity or resistance of a particular patient's tumor to a chemotherapeutic agent or combinations of chemotherapeutic agents.

A broad variety of chemotherapeutic agents are used in the treatment of human cancer. These include the plant alkaloids vincristine, vinblastine, vindesine, and VM-26; the antibiotics actinomycin-D, doxorubicin, daunorubicin, mithramycin, mitomycin C and bleomycin; the antimetabolites methotrexate, 5-fluorouracil, 5-fluorodeoxyuridine, 6-mercaptopurine, 6-thioguanine, cytosine arabinoside, 5-aza-cytidine and hydroxyurea; the alkylating agents cyclophosphamide, melphalan, busulfan, CCNU, MeCCNU, BCNU, streptozotocin, chlorambucil, bis-diamminedichloroplatinum, azetidinylbenzoquinone; and the miscellaneous agents dacarbazine, mAMSA and mitoxantrone (DeVita et al., Id.). However, some neoplastic cells become resistant to specific chemotherapeutic agents, in some instances even to multiple chemotherapeutic agents, and some tumors are intrinsically resistant to certain chemotherapeutic agents. Such drug resistance or multiple drug resistance can theoretically arise from expression of genes that confer resistance to the agent, or from lack of expression of genes that make the cells sensitive to a particular anticancer drug. One example of the former type is the multidrug resistance gene, MDR1, which encodes an integral plasma membrane protein termed P-glycoprotein that is a non-specific, energy-dependent efflux pump. (See Roninson (ed)., 1991, Molecular and Cellular Biology of Multidrug Resistance in Tumor Cells, Plenum Press, N.Y., 1991; Gottesman et al., 1991, in Biochemical Bases for Multidrug Resistance in Cancer, Academic Press, N.Y., Chapter 11 for reviews). Examples of the latter type include topoisomerase II, the expression of which makes cells sensitive to the anticancer drug etoposide. Decreased expression of this enzyme makes neoplastic cells resistant to this drug. (See Gudkov et al., 1993, Proc. Natl. Acad. Sci. USA 90: 3231-3235). Although these are just single examples of the way that modulation of gene expression can influence chemotherapeutic drug sensitivity or resistance in neoplastic cells, these examples demonstrate the diagnostic and prognostic potential for identifying genes the expression of which (or the pattern of gene expression modulation thereof) are involved in mediating the clinical effectiveness of anticancer drug treatment.

Breast cancer continues to be a significant public health problem, with one of eight women affected by this disease during their lifetime. More than 190,000 women will be diagnosed with breast cancer in 2003. Although metastatic breast cancer remains an incurable disease, improved patient survival has resulted from the recent addition of the taxanes paclitaxel and docetaxel to treatment regimens commonly employed for breast cancer. However, these drugs are not without side effects. Both agents cause the blood counts to drop, leading to increased risk of infection, and they cause nerve damage resulting in numbness in the hands and feet that can make it difficult for some patients to carry out their routine activities. While these agents are active, not all women benefit. The percentage of women who have tumors that shrink substantially after receiving a taxane ranges from 25% to 50%. There remains a need for methods of identifying those women who would benefit from taxane treatment and those who would not. A genetic test performed on a woman's cancer specimen that could readily identify those women who are most likely to respond would be of great value. Similarly, a test that that could identify those women with taxane resistant tumors would make it possible for them to avoid potential drug related toxicity without an associated anticancer benefit. See, for example, Nabholtz et al., 2003, Clin Breast Cancer. 4:187-92; Davidson, 2002, Clin Breast Cancer., 3 Suppl 2:S53-8; Piccart, 2003, Breast Cancer Res Treat. 79 Suppl 1:S25-34; Valero, 2002, Oncology (Huntingt). 16(6 Suppl 6):35-43.

Thus, there is a need in this art for developing methods for identifying genes and gene expression patterns that are predictive of the clinical effectiveness of anticancer drug treatment therapies, in order to make more informed decisions for treating individual cancer patients with anticancer drugs having greatest likelihood of producing a positive outcome.

### SUMMARY OF THE INVENTION

The present invention provides methods identifying genes and gene expression patterns that are predictive of the clinical effectiveness of anticancer drug treatment therapies.

In a first aspect, the invention provides methods for identifying genes having an expression pattern in tumor cells that is modulated when the cells are contacted with a chemotherapeutic agent comprising the steps of:

a) separating living neoplastic cells from dead cells, vascular endothelial cells and living stromal cells in a mixed population of cells from a tumor sample, by

i) contacting the mixed population of cells with a vital stain or fluorescent dye;

ii) contacting the mixed population of cells with a detectably-labeled immunological reagent that specifically binds to neoplastic cells; and

iii) selecting the cells in the mixed population of step (b) that are not stained with the vital stain and that bind the immunological reagent,

b) contacting the separated, living neoplastic cells with a chemotherapeutic amount of a chemotherapeutic agent;

c) separating apoptotic and non-apoptotic cells of step b) by contacting the cells with a reagent specific for apoptosis and sorting the population of cells in step b) thereby;

d) assaying gene expression in each of the separated populations of apoptotic and non-apoptotic cells; and

e) identifying genes having an expression pattern that is modulated by contacting cells with a chemotherapeutic amount of a chemotherapeutic agent.

In preferred embodiments, expression of one or a plurality of genes is increased in cells sensitive to the chemotherapeutic agent. In these embodiments, the genes are Sjogren syndrome antigen B (autoantigen La), capping protein alpha, adenine nucleotide translocator 3 (liver), AU-rich element RNA-binding protein AUF1, phosphatidylserine synthase I, integrin, alpha L, lymphocyte function-associated antigen 1, branched chain keto acid dehydrogenase E1, alpha polypeptide, annexin XI (56 kD autoantigen), or Von Hippel-Lindau syndrome. In alternative embodiments, expression of one or a plurality of genes is increased in cells resistant to the chemotherapeutic agent. In these embodiments, the genes are myosin phosphatase target subunit 1 (MYPT1), albumin D-box binding protein, complement component 7, plasminogen activator, urokinase receptor, ATPase, DNA binding protein (HIP 116), zinc finger protein (ZNF198) or tropomodulin. As provided herein, the invention discloses said genes differentially expressed in drug resistant and drug sensitive breast cancer tumor cells. More particularly, said differential gene expression is detected in breast cancer tumor cells that are resistant or sensitive to taxane chemotherapeutic drugs, including taxol, paclitaxol and docetaxol.

In a second aspect, the invention provides methods for identifying one or a plurality of genes having a pattern of expression that is different in a tumor cell sensitive to a chemotherapeutic drug than the expression pattern in a tumor cell resistant to the chemotherapeutic drug, the method comprising the steps of:

a) performing an extreme drug resistance (EDR) assay on a mixed population of cells from a tumor sample;

b) separating living tumor cells from dead cells, vascular endothelial cells and living stromal cells in the mixed population of cells from a tumor sample, by

i) contacting the mixed population of cells with a vital stain or fluorescent dye;

ii) contacting the mixed population of cells with a detectably-labeled immunological reagent that specifically binds to neoplastic cells; and

iii) selecting the cells in the mixed population that are not stained with the vital stain and that bind the immunological reagent,

c) assaying gene expression in each of the separated populations of drug sensitive and drug resistant cells; and

d) identifying genes having an expression pattern that is different in the drug resistant cells than in the drug sensitive cells.

In preferred embodiments, expression of one or a plurality of genes is increased in cells sensitive to the chemotherapeutic agent. In these embodiments, the genes are Sjogren syndrome antigen B (autoantigen La), capping protein alpha, adenine nucleotide translocator 3 (liver), AU-rich element RNA-binding protein AUF1, phosphatidylserine synthase I, integrin, alpha L, lymphocyte function-associated antigen 1, branched chain keto acid dehydrogenase E1, alpha polypeptide, annexin XI (56 kD autoantigen), or Von Hippel-Lindau syndrome. In alternative embodiments, expression of one or a plurality of genes is increased in cells resistant to the chemotherapeutic agent. In these embodiments, the genes are myosin phosphatase target subunit 1 (MYPT1), albumin D-box binding protein, complement component 7, plasminogen activator, urokinase receptor, ATPase, DNA binding protein (HIP 116), zinc finger protein (ZNF198) or tropomodulin. As provided herein, the invention discloses said genes differentially expressed in drug resistant and drug sensitive breast cancer tumor cells. More particularly, said differential gene expression is detected in breast cancer tumor cells that are resistant or sensitive to taxane chemotherapeutic drugs, including taxol, paclitaxol and docetaxol.

In a third aspect, the invention provides a pattern of gene differential gene expression, comprising increased expression of one or a plurality of genes that are myosin phosphatase target subunit 1 (MYPT1), albumin D-box binding protein, complement component 7, plasminogen activator, urokinase receptor, ATPase, DNA binding protein (HIP 116), zinc finger protein (ZNF198) or tropomodulin, wherein increased expression of said one or plurality of genes in a tumor cell compared with a non-tumor cell identifies said tumor cell to be a cell resistant to chemotherapeutic drugs that are taxanes. In addition, in this aspect the invention provides a pattern of gene differential gene expression, comprising increased expression of one or a plurality of genes that are Sjogren syndrome antigen B (autoantigen La), capping protein alpha, adenine nucleotide translocator 3 (liver), AU-rich element RNA-binding protein AUF1, phosphatidylserine synthase I, integrin, alpha L, lymphocyte function-associated antigen 1, branched chain keto acid dehydrogenase E1, alpha polypeptide, annexin XI (56 kD autoantigen), or Von Hippel-Lindau syndrome wherein increased expression of said one or plurality of genes in a tumor cell compared with a non-tumor cell identifies said tumor cell to be a cell sensitive to chemotherapeutic drugs that are taxanes.

Accordingly, the invention in its fourth aspect provides methods for identifying a tumor or cells comprising the tumor that are resistant to taxane chemotherapeutic drugs, the method comprising the steps of:

a) determining gene expression levels in a tumor sample or cells comprising the tumor for one or a plurality of genes that are myosin phosphatase target subunit 1 (MYPT1), albumin D-box binding protein, complement component 7, plasminogen activator, urokinase receptor, ATPase, DNA binding protein (HIP 116), zinc finger protein (ZNF198) or tropomodulin;

b) comparing gene expression levels of the one or plurality of genes in step a) with gene expression levels of said one or plurality of genes in a non-tumor sample or cells comprising said sample; and

c) identifying the tumor or cells comprising the tumor to be resistant to taxane chemotherapeutic drugs when the gene expression levels of one or a plurality of said genes is increased in the tumor sample when compared to gene expression levels in the non-tumor sample.

Most preferably, the tumor or cells comprising the tumor are breast cancer tumors or cells thereof. In an alternative embodiment of this aspect, the invention provides methods for identifying a tumor or cells comprising the tumor that are sensitive to taxane chemotherapeutic drugs, the method comprising the steps of:

a) determining gene expression levels in a tumor sample or cells comprising the tumor for one or a plurality of genes that are Sjogren syndrome antigen B (autoantigen La), capping protein alpha, adenine nucleotide translocator 3 (liver), AU-rich element RNA-binding protein AUF1, phosphatidylserine synthase I, integrin, alpha L, lymphocyte function-associated antigen 1, branched chain keto acid dehydrogenase E1, alpha polypeptide, annexin XI (56 kD autoantigen), or Von Hippel-Lindau syndrome;

b) comparing gene expression levels of the one or plurality of genes in step a) with gene expression levels of said one or plurality of genes in a non-tumor sample or cells comprising said sample; and

c) identifying the tumor or cells comprising the tumor to be sensitive to taxane chemotherapeutic drugs when the gene expression levels of one or a plurality of said genes is increased in the tumor sample when compared to gene expression levels in the non-tumor sample.

Most preferably, the tumor or cells comprising the tumor are breast cancer tumors or cells thereof. In all the embodiments of these methods of the invention, the taxane chemotherapeutic drug is preferably taxol, paclitaxol or docetaxol.

In a fifth aspect, the invention provides methods for detecting a gene expression profile of living neoplastic cells that are resistant to a taxane cytotoxic compound and distinguishing said profile from the gene expression profile of living neoplastic cells that are sensitive to the taxane cytotoxic compound in a mixed population of cells from a tumor sample, the method comprising the steps of:

a) contacting the mixed population of cells with the taxane cytotoxic compound for a time and at a concentration wherein the neoplastic cells that are sensitive to the taxane cytotoxic compound undergo apoptosis;

b) contacting the mixed population of step (a) with a vital stain or fluorescent dye;

c) contacting the mixed population of cells of step (b) with a discrimination compound that specifically binds to apoptotic cells;

d) contacting the mixed cell population of step (c) with a detectably-labeled immunological reagent that specifically binds to the apoptotic cell discrimination compound; and

e) separating the cells in the mixed population of step (d) that are not stained with the vital stain from the cells that are stained with the vital stain;

f) separating the cells in the mixed population of step (e) that are not stained with the vital stain and that do not bind the immunological reagent from the cells in the mixed population of step (c) that are not stained with the vital stain and that do bind the immunological reagent;

g) isolating cellular RNA from the each of the separated cells selected in step

h) preparing detectably-labeled cDNA from the cellular RNA isolated in step (g);

i) hybridizing each of the cDNA preparations prepared in step (h) to a gene array comprising at least 4000 eukaryotic genes;

j) detecting a pattern of gene expression for hybridization of each of the cDNA preparations prepared from the mRNA isolated from the cells selected in step (f); and

k) comparing the pattern of gene expression detected in step (j) from hybridization of the microarray with cDNA from cells that are not stained with the vital stain and that do not bind the immunological reagent with a pattern of gene expression obtained by hybridizing cDNA prepared from cells that are not stained with the vital stain and that do bind the immunological reagent.

As practiced according to the teachings herein, the vital stain is preferably propidium iodide, and the discrimination compound is Annexin V. In advantageous embodiments, the immunological reagent specifically binds to Annexin V and is detectably labeled with a fluorescent label. Preferably, the cells of step (f) are selected by fluorescence-activated cell sorting. Taxane compounds useful in the practice of this invention include paclitaxol, taxol or docetaxol. Advantageously, cDNA used in the practice of this aspect of the methods of the invention is detectably labeled with a fluorescent label. Preferably, the mixed population is contacted with the taxane cytotoxic compound under in vitro cell culture conditions whereby the cells cannot attach to a solid substrate. Most preferably, the tumor or cells comprising the tumor are breast cancer tumors or cells thereof.

As practiced according to the methods of the invention, preferred vital stains include propidium iodide, fast green dyes and trypan blue. In addition, immunological reagents used according to the methods of the invention are preferably detectably labeled with a fluorescent label. Said immunological reagents are preferably antibodies, more preferably tumor-specific antibodies, and most preferably antibodies antibody is immunologically specific for EGFR or HER2. Detection, discrimination and separation of drug-sensitive and drug-resistant cells according to the methods of the invention are preferably accomplished by cell sorting, most preferably by fluorescence-activated cell sorting.

The methods of the invention are preferably performed using a solid tumor sample that is a breast cancer sample, most preferably a disaggregated breast cancer tumor sample.

It is an advantage of the methods of this invention that homogeneous neoplastic cell populations from breast cancer tumors, both malignant and benign, can be obtained separated from stromal cells, infiltrating non-neoplastic hematopoietic cells and other tumor components. This feature of the inventive methods are advantageous because the presence of such contaminating, non-neoplastic cells in tumor sample preparations confounds analyses directed at detecting neoplastic cell-specific properties, such as patterns of gene expression as disclosed herein. It is also an advantage of the present inventive methods that drug-resistant and drug-sensitive neoplastic cells can be separated from homogeneous breast cancer tumor cell populations. As a result, RNA preparations specific for drug-resistant and drug-sensitive breast cancer cells are obtained that can be used to identify genes, and patterns of genes, that are differentially expressed in drug-resistant and drug-sensitive tumor cells. In addition, the methods of the invention as provided permit drug-resistant and drug-sensitive tumor cells to be simultaneously treated with cytotoxic drugs in the same mixed cell culture, thereby avoiding experimental variability.

In another aspect, the invention provides methods for identifying one or a plurality of genes having a pattern of expression that is different in a tumor cell sensitive to a chemotherapeutic drug than the expression pattern in a tumor cell resistant to the chemotherapeutic drug, the method comprising the steps of:

a) performing an extreme drug resistance (EDR) assay on a mixed population of cells from a tumor sample;

b) separating living malignant cells from nonmalignant cells in the mixed population of cells from a tumor sample, by contacting the mixed population of cells with a detectably-labeled immunological reagent that specifically binds to malignant cells;

c) assaying gene expression in each of the separated populations of the malignant cells; and

d) identifying genes having an expression pattern that is different in the drug resistant cells than in the drug sensitive cells.

In another aspect, the invention provides methods for identifying genes having an expression pattern in tumor cells that is modulated when the cells are contacted with a chemotherapeutic agent comprising the steps of:

a) separating malignant cells from nonmalignant cells in a mixed population of cells from a tumor sample, by

i) contacting the mixed population of cells with a detectably-labeled immunological reagent that specifically binds to malignant cells; and

ii) sorting the cells, for example, by flow cytometry or immunomagnetic beads;

b) contacting the separated, living malignant cells with a chemotherapeutic amount of a chemotherapeutic agent, such as a taxane cytotoxic compound;

c) separating apoptotic and non-apoptotic cells of step b) by contacting the cells with a reagent specific for apoptosis and sorting the population of cells in step b) thereby;

d) assaying gene expression in each of the separated populations of apoptotic and non-apoptotic cells; and

e) identifying genes having an expression pattern that is modulated by contacting cells with a chemotherapeutic amount of a chemotherapeutic agent.

In one embodiment of the invention, the preceding two aspects can be combined in one method.

In another aspect, the invention provides methods for detecting a gene expression profile of malignant cells that are resistant to a taxane cytotoxic compound and distinguishing said profile from the gene expression profile of malignant cells that are sensitive to the taxane cytotoxic compound in a mixed population of cells from a tumor sample, the method comprising the steps of separating malignant cells into a purified population of cells:

a) contacting a purified population of cells with the taxane cytotoxic compound for a time and at a concentration wherein the malignant cells that are sensitive to the taxane cytotoxic compound undergo apoptosis;

b) contacting the purified population of step (a) with a vital stain or fluorescent dye;

c) contacting the purified population of cells of step (b) with a discrimination compound that specifically binds to apoptotic cells;

d) contacting the purified cell population of step (c) with a detectably-labeled immunological reagent that specifically binds to the apoptotic cell discrimination compound; and

e) separating the cells in the purified population of step (d) that are not stained with the vital stain from the cells that are stained with the vital stain;

f) separating the cells in the purified population of step (e) that are not stained with the vital stain and that do not bind the immunological reagent from the cells in the purified population of step (c) that are not stained with the vital stain and that do bind the immunological reagent;

g) isolating cellular RNA from the each of the separated cells selected in step (f);

h) preparing detectably-labeled cRNA from the cellular RNA isolated in step (g);

i) hybridizing each of the cRNA preparations prepared in step (h) to a gene array comprising at least 4000 eukaryotic genes;

j) detecting a pattern of gene expression for hybridization of each of the cRNA preparations prepared from the mRNA isolated from the cells selected in step (f); and

k) comparing the pattern of gene expression detected in step (j) from hybridization of the microarray with cRNA from cells that are not stained with the vital stain and that do not bind the immunological reagent with a pattern of gene expression obtained by hybridizing cRNA prepared from cells that are not stained with the vital stain and that do bind the immunological reagent.

Specific preferred embodiments of the present invention will become evident from the following more detailed description of certain preferred embodiments and the claims.

### BRIEF DESCRIPTION OF THE DRAWINGS

FIG. 1 is a schematic flowchart illustrating an embodiment of the methods of the invention showing how drug-resistant neoplastic cell-specific mRNA is used to probe a gene expression microarray.

FIG. 2 is a schematic flowchart illustrating an embodiment of the methods of the invention showing the Extreme Drug Resistance® Assay used for preparing tumor explants for cell sorting analysis.

FIG. 3 shows fluorescence-activated cell sorting (FACS) profiles of breast cancer explant-derived tumor cells showing the mixed population of EGFR⁺ and EGFR⁻ cells (top), and the population sorted into EGFR⁺ (bottom right) and EGFR⁻ (bottom left) cell populations.

FIG. 4 is a schematic diagram showing FACS sorting of breast cancer tumor-derived vascular endothelial cells, sorted by binding to CD31- and CD105-specific antibodies, and verified by immunohistochemical staining.

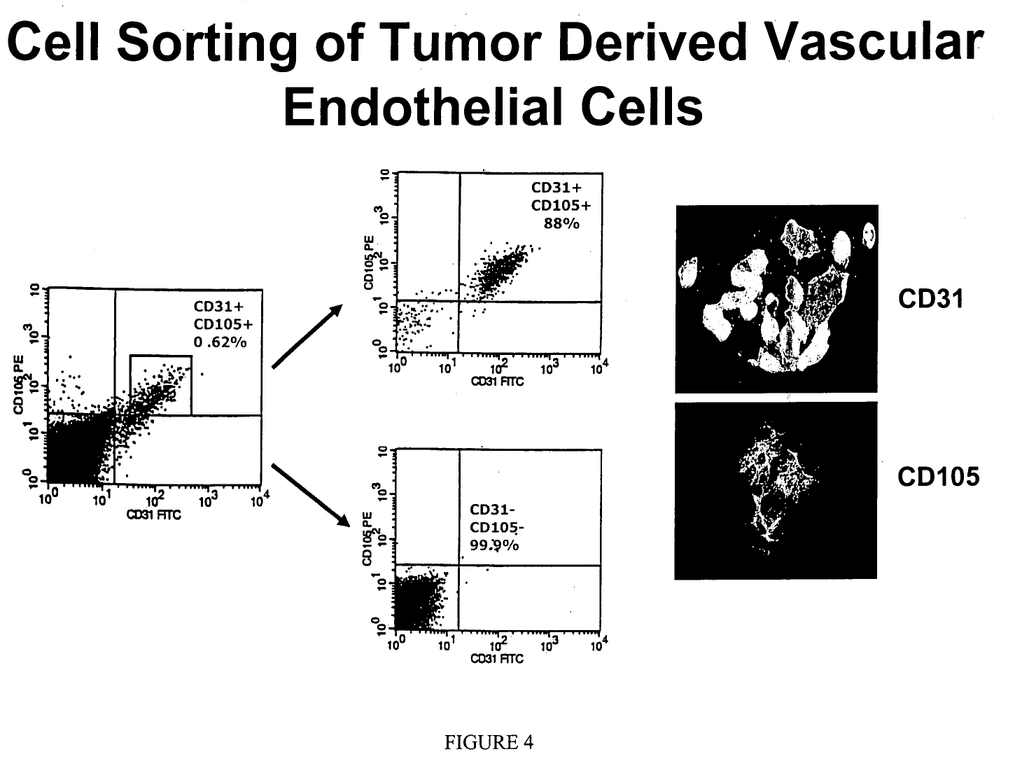

FIG. 5 is a schematic diagram of Annexin V detection of apoptosis used in negatively selecting apoptotic, drug sensitive breast cancer tumor cells.

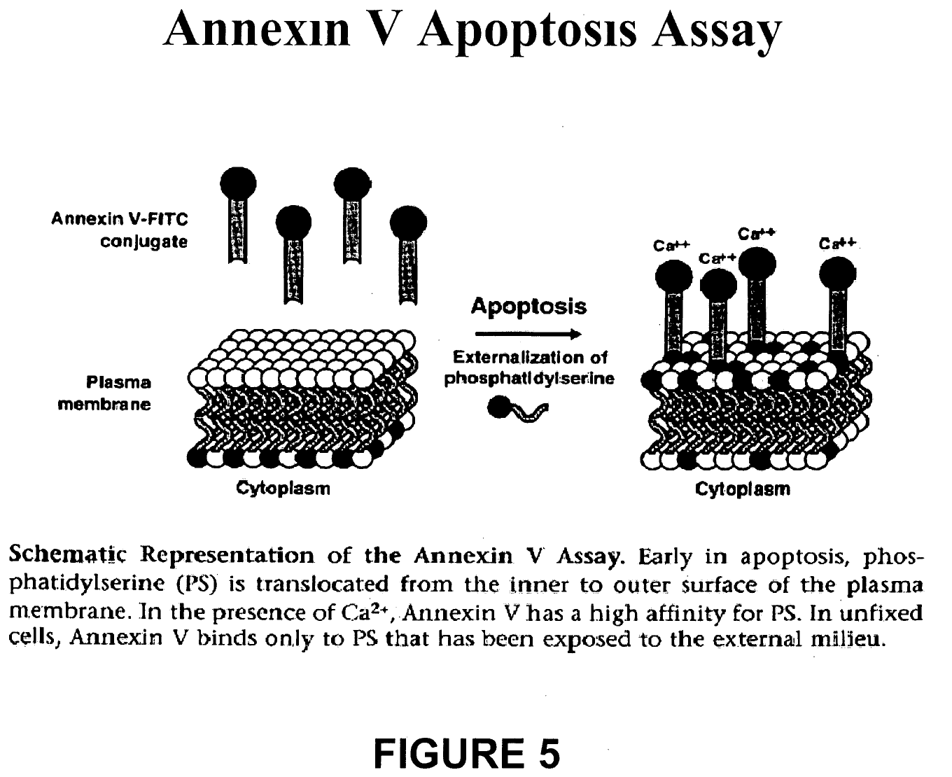

FIG. 6 are fluorescence-activated cell sorting (FACS) profiles of taxane sensitive and—resistant cells from breast cancer tumor explants after in vitro cytotoxic drug treatment. FIG. 6 (top) shows Annexin V/propidium iodide discrimination of the cells into apoptotic (Annexin V+) and non-apoptotic (Annexin V−) populations, that can be sorted into pure populations of each (bottom). The Annexin V+population is comprised of both apoptotic, drug-sensitive breast cancer tumor cells and dying, drug-sensitive breast cancer tumor cells, while the Annexin-population is comprised of living, drug-resistant breast cancer tumor cells.

FIG. 7 illustrates the results of hierarchical clustering/Spearman rank correlation of results obtained using ResGen™ GenFilters® as disclosed in Example 2.

FIG. 8 is a schematic diagram showing Venn Diagram statistical analysis of the overlap in differentially-expressed genes obtained for taxane-sensitive and taxane-resistant human breast cancer tumor cells, human breast cancer tumor-derived vascular endothelial cells, and human breast cancer cell lines.

### DETAILED DESCRIPTION OF THE PREFERRED EMBODIMENTS

The present invention provides a method for making a prognosis about disease course in a human cancer patient. For the purposes of this invention, the term “prognosis” is intended to encompass predictions and likelihood analysis of disease progression, particularly tumor recurrence, metastatic spread and disease relapse. The prognostic methods of the invention are intended to be used clinically in making decisions concerning treatment modalities, including therapeutic intervention, diagnostic criteria such as disease staging, and disease monitoring and surveillance for metastasis or recurrence of neoplastic disease.

The methods of the invention are preferably performed using human cancer patient tumor samples, most preferably samples from patients with breast cancer. Such samples can be, inter alia, biopsy or surgical specimens taken directly from a human breast cancer patient, or samples preserved, for example in paraffin, and prepared for histological and immunohistochemical analysis.

For the purposes of this invention, the term “tumor sample” is intended to include resected solid tumors, biopsy material, and pathological specimens, as well as benign tumors, particularly tumors of certain tissues such as brain and the central nervous system. As disclosed herein, preferred tumor samples according to the invention are breast cancer tumor samples. One of ordinary skill will appreciate that samples derived from solid tumors, such as breast cancer, will require combinations of physical and chemical/enzymatic disaggregation to separate neoplastic cells from stromal cells and infiltrating hematopoietic cells.

In the practice of the methods of this invention, living cells are separated from dying cells, dead cells and cell debris, and drug sensitive and drug resistant cells are separated from each other and from non-neoplastic cells according to the methods of the invention by cell sorting methods, most preferably fluorescence-activated cell sorting (FACS). Separation of living cells from dying cells, dead cells and cell debris is facilitated by contacting mixed cell populations with a vital stain, preferably a fluorescent vital stain, such as propidium iodide (PI) and ethidium bromide (EtBr). Separation of drug sensitive and drug resistant cells from one another and from non-neoplastic cells using reagents, most preferably immunological agents, that discriminate between such cells. In particular, drug resistant neoplastic cells are separated from drug sensitive neoplastic cells after incubation with a cytotoxic amount of a chemotherapeutic drug by contacting the mixed cell population with a discrimination compound that specifically binds to apoptotic cells, and separation is achieved using reagents, most preferably immunological agents, that specifically binds to the discrimination compound. In preferred embodiments, the discrimination compound is an annexin, most preferably annexin V or antibodies directed against caspases.

As used herein, the term “homogeneous collection” is intended to describe tumor samples, either after enrichment or as obtained directly from a patient sample, wherein a majority of the cells are tumors cells, more preferably comprising at least 70%, 75%, 80%, 85% or 90% tumor cells as determined by histological examination, in vitro growth capacity, or expression of a tumor-specific marker gene.

For the purposes of this invention, the term “immunological reagents” is intended to encompass antisera and antibodies, particularly monoclonal antibodies, as well as fragments thereof (including F(ab), F(ab)₂, F(ab)N and Fᵥ fragments). Also included in the definition of immunological reagent are chimeric antibodies, humanized antibodies, and recombinantly-produced antibodies and fragments thereof, as well as aptamers (i.e., oligonucleotides capable of interacting with target molecules such as peptides). Immunological methods used in conjunction with the reagents of the invention include direct and indirect (for example, sandwich-type) labeling techniques, immunoaffinity columns, immunomagnetic beads, fluorescence activated cell sorting (FACS), enzyme-linked immunosorbent assays (ELISA), and radioimmune assay (RIA), most preferably FACS. For use in these assays, the neoplastic immunological reagents can be labeled, using fluorescence, antigenic, radioisotopic or biotin labels, among others, or a labeled secondary or tertiary immunological detection reagent can be used to detect binding of the neoplastic immunological reagents (i.e., in secondary antibody (sandwich) assays).

Examples of immunological reagents useful in the practice of this invention include antibodies, most preferably monoclonal antibodies that recognize tumor antigens such as CA15-3, HER2, and EGFR (all of which recognize cellular epitopes in breast cancer cells).

The immunological reagents of the invention are preferably detectably-labeled, most preferably using fluorescent labels that have excitation and emission wavelengths adapted for detection using commercially-available instruments such as and most preferably fluorescence activated cell sorters. Examples of fluorescent labels useful in the practice of the invention include phycoerythrin (PE), fluorescein isothiocyanate (FITC), rhodanine (RH), Texas Red (TX), Cy3, Hoechst 33258, and 4′,6-diamidino-2-phenylindole (DAPI). Such labels can be conjugated to immunological reagents, such as antibodies and most preferably monoclonal antibodies using standard techniques (Maino et al., 1995, Cytometry 20: 127-133).

As used herein, the terms “microarray,” “bioarray,” “biochip” and “biochip array” refer to an ordered spatial arrangement of immobilized biomolecular probes arrayed on a solid supporting substrate. Preferably, the biomolecular probes are immobilized on second linker moieties in contact with polymeric beads, wherein the polymeric beads are immobilized on first linker moieties in contact with the solid supporting substrate. Biochips, as used in the art, encompass substrates containing arrays or microarrays, preferably ordered arrays and most preferably ordered, addressable arrays, of biological molecules that comprise one member of a biological binding pair. Typically, such arrays are oligonucleotide arrays comprising a nucleotide sequence that is complementary to at least one sequence that may be or is expected to be present in a biological sample. Alternatively, and preferably, proteins, peptides or other small molecules can be arrayed in such biochips for performing, inter alia, immunological analyses (wherein the arrayed molecules are antigens) or assaying biological receptors (wherein the arrayed molecules are ligands, agonists or antagonists of said receptors). Useful microarrays for detecting differential gene expression between chemotherapeutic drug sensitive and resistant neoplastic cells are described, inter alia, in U.S. Pat. No. 6,040,138 to Lockhart et al. (commercially-available from Affymetrix, Inc., Santa Clara, Calif.) and U.S. Pat. No. 6,004,755 to Wang (commercially-available from Incyte Inc., Palo Alto, Calif.) and are also commercially available, inter alia, from Research Genetics (Huntsville, Ala.).

As used in the methods of the invention, gene arrays or microarrays comprise of a solid substrate, preferably within a square of less than about 10 microns by 10 microns on which a plurality of positionally-distinguishable polynucleotides are attached. These probe sets can be arrayed onto areas of up to 1 to 2 cm², providing for a potential probe count of >30,000 per chip. The solid substrate of the gene arrays can be made out of silicon, glass, plastic or any suitable material. The form of the solid substrate may also vary and may be in the form of beads, fibers or planar surfaces. The sequences of these polynucleotides are determined from tumor-specific gene sets identified by analysis of gene expression profiles from a plurality of tumors as described above. The polynucleotides are attached to the solid substrate using methods known in the art (see, for example, DNA MICROARRAYS: A PRACTICAL APPROACH, Schena, ed., Oxford University Press: Oxford, UK, 1999) at a density at which hybridization of particular polynucleotides in the array can be positionally distinguished. Preferably, the density of polynucleotides on the substrate is at least 100 different polynucleotides per cm², more preferably at least 300 polynucleotides per cm². In addition, each of the attached polynucleotides comprises at least about 25 to about 50 nucleotides and has a predetermined nucleotide sequence. Target RNA or cDNA preparations are used from tumor samples that are complementary to at least one of the polynucleotide sequences on the array and specifically bind to at least one known position on the solid substrate.

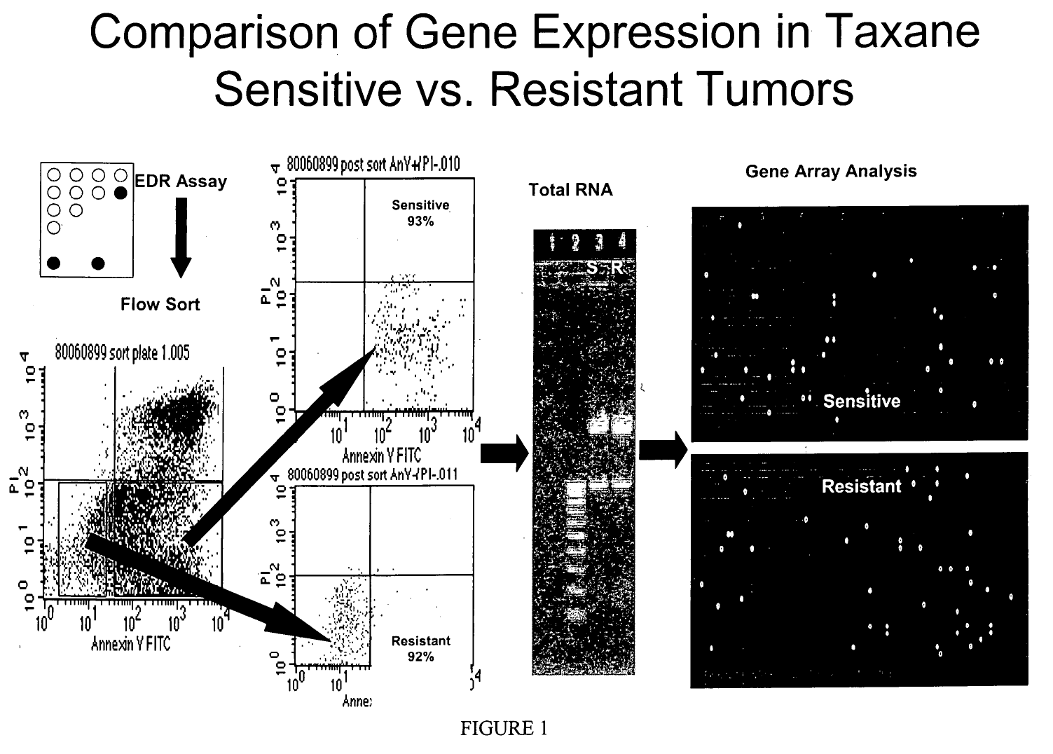

The practice of one embodiment of the invention is shown in FIG. 1. Resistance and sensitivity to a specific class of chemotherapeutic drug, taxanes, of breast tumor cells is determined by analyzing differential gene expression. As disclosed herein, resistance or sensitivity can be intrinsic, i.e. a property of the cell prior to exposure to taxanes, or induced by exposure of breast cancer cells to drug.

A breast tumor sample or a breast tumor cell line is harvested and pure cancer cell population obtained by FACS sorting using fluorescently-labeled antibodies specific for neoplastic cell markers specific for breast cancer cells (such as HER2 or EGFR). The sorted pure cancer cell population is then expanded by growth in cell culture to provide sufficient cells for separation into drug-sensitive and drug-resistant populations. Drug resistant cells are separated from drug sensitive cells by culture in increasing concentrations of cytotoxic drugs, and the degree of drug resistance quantitated by growing the cells in a cell proliferation-specific detectable label (such as tritiated thymidine) for a terminal portion of each cell culture experiment. IC₅₀ values can be established by performing this assay in cytophobic plates that inhibit cell attachment (and therefore prevent proliferation of non-neoplastic cells). Finally, cell culture at the IC₅₀ concentration of the cytotoxic drug in cytophobic plates is used to prepare neoplastic cells for flow sorting. It will be recognized that a significant advantage of these methods is that a mixed population of drug-sensitive and drug-resistant cells are treated simultaneously under exactly identical conditions of cell culture and drug treatment and then analyzed after separation based on their differential drug resistance characteristics.

Drug sensitive neoplastic cells are separated from drug resistant neoplastic cells, most preferably using fluorescence-activated cell sorting. Cells cultured in cytotoxic drug at the IC₅₀ are stained with a fluorescent vital stain such as propidium iodide and contacted with an apoptosis-specific, discrimination compound and with a fluorescently-labeled immunological reagent that specifically labels the apoptotic, drug sensitive neoplastic cells. In a preferred embodiment, the discrimination reagent Annexin V, which binds to phosphatidylserine exposed by apoptosis in drug sensitive cells and does not bind to drug resistant neoplastic cells. FACS analysis separates the drug resistant, living cells from cell debris, dead cells (such as stromal cells) and drug-sensitive neoplastic cells. It is also an advantage of the inventive methods that FACS sorting can discriminate between drug sensitive neoplastic cells (typically caused to be apoptotic as a result of cytotoxic drug treatment), drug resistant neoplastic cells and dead or dying cells by gating the cell sorter to perform simultaneous discrimination between these different components of the mixed population.

Cell sorting according to the methods of the invention provides sufficient numbers of separated drug-sensitive and drug-resistant neoplastic cells to be able to perform gene expression analysis. Gene expression analysis is performed to detect differences in gene expression between pure populations of neoplastic cells that are sensitive to a cytotoxic, chemotherapeutic drug such as taxane and drug resistant neoplastic cells. RNA from the drug resistant neoplastic cells and drug sensitive neoplastic cells separated, most preferably, by FACS sorting is individually isolated and cDNA prepared therefrom. In preferred embodiments, the cDNA is detectably labeled, for example using radioactively-labeled or fluorescently-labeled nucleotide triphosphates. Hybridization of gene expression microarrays produces pattern of gene expression specific for cytotoxic, chemotherapeutic drug resistant neoplastic cells and neoplastic cells sensitive to the same drug and derived from the same cytotoxic drug-treated mixed cell population from which the drug-resistant cells were obtained. Identification of genes and patterns of genes differentially expressed in these cells is established by comparison of the gene expression pattern obtained by performing the microarray hybridization analysis on cDNA from neoplastic cells that are resistant to and sensitive to the cytotoxic, chemotherapeutic drug. Advantageously, tumor samples from human patients and taxane-resistant and -sensitive breast cancer cell lines are compared using bioinformatics analysis to identify genes statistically correlated with drug resistance or sensitivity. Additionally, tumor-derived vascular endothelial cells (VECs) separated by cell sorting methods using VEC-specific antibodies are also analyzed and compared to both human breast cancer patient samples and taxane-resistant and -sensitive breast cancer cell lines. Gene expression patterns specific for taxane-resistant and -sensitive breast cancer cells are thus obtained using the inventive methods.

As used herein, an “extreme drug resistance assay” (EDR assay) refers to assays performed on tumor specimens without prior drug exposure to define intrinsic drug resistance. Gene arrays on cancer cells sorted from a tumor prior to drug exposure reflect a static, constitutive expression profile. An EDR status in the assay can be correlated to these static genes and transcript profiles can classify the tumors as LDR, IDR or EDR.

Differences in tumor genetics within a patient's cancer and between patients leads to heterogeneity of drug response. Such differences challenge the oncologist's ability to select the most appropriate drugs for each patient. Clinical trials may identify the most active agents in a group of breast or ovarian cancer patients, but without specific testing on a given patient's cancer one cannot discriminate the responders from the non-responders in advance of trying the drug in the patient. Custom gene arrays made up of sets of genes discovered through the processes described herein can be used to identify taxane resistant and sensitive tumors prior to treatment, making it possible to tailor the selection of drugs to each patient's genetic expression pattern.

The following Examples are intended to further illustrate certain preferred embodiments of the invention and are not limiting in nature.

### EXAMPLE 1

### Tumor Specimen Handling

Viable breast tumor samples were obtained from patients with malignant disease and placed into Oncotech transport media (complete medium, RPMI supplemented with 3% Fetal Calf Serum and antibiotics, as described below in the section Tissue Culture and Expansion) by personnel at the referring institution immediately after collection and shipped to Oncotech by overnight courier for the purpose of determining the tumors in vitro drug response profile. Upon receipt, data on tissue diagnosis, treatment history, referring physician, and patient information about the specimen was entered into a computer database. The tumor was then processed by the laboratory where three areas of the tumor are removed from the sample, fixed in Formalin, paraffin embedded, sectioned and Hematoxylin and eosin stained for pathologists' review to ensure agreement with the referring institution histological diagnosis. After in vitro drug response of the tumor specimens were determined by the laboratory, this information was sent back to the treating physician to aid in their treatment selection.

The remainder of the sample is disaggregated mechanically and processed into a cell suspension for the Extreme Drug Resistance (EDR) assay. A cytospin preparation from a single cell suspension of the tumor was examined by a technologist to determine the presence and viability of malignant cells in the specimen.

### EDR Assay

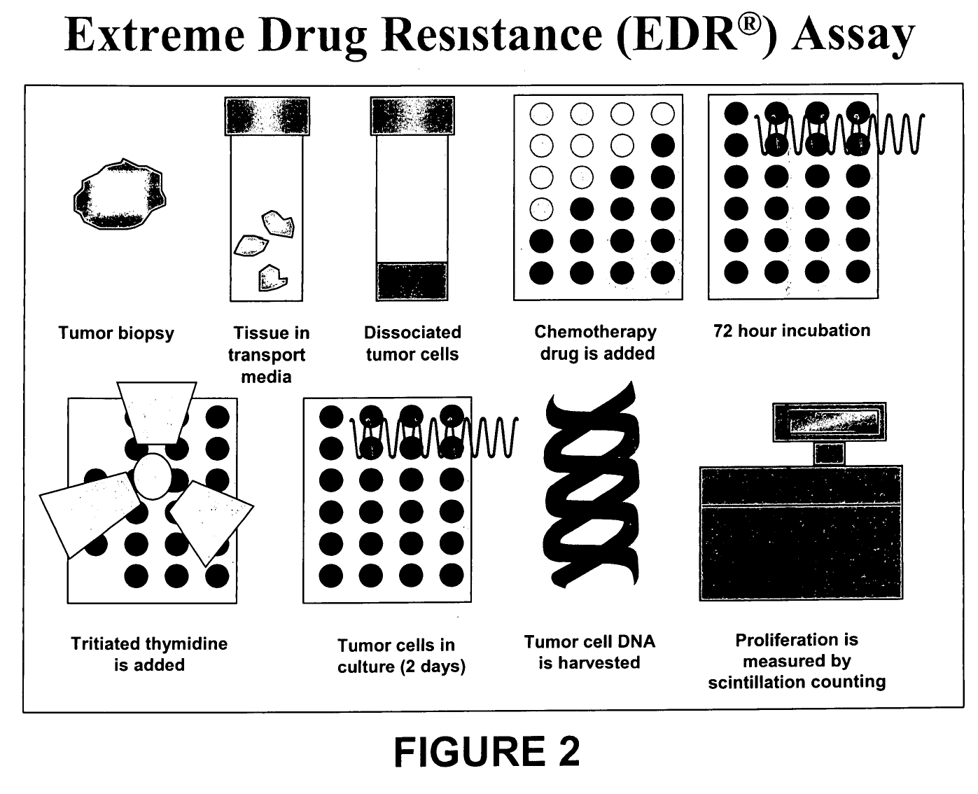

The EDR assay is an agarose-based culture system, using tritiated thymidine incorporation to define in vitro drug response; a schematic diagram of this assay is shown in FIG. 2. This assay is predictive of clinical response (Kern et al., 1990, “Highly specific prediction of antineoplastic resistance with an in vitro assay using suprapharmacologic drug exposures,” J. Nat. Cancer Inst. 82: 582-588). Tumors were cut with scissors into pieces of 2 mm or smaller in a Petri dish containing 5 mL of complete medium. The resultant slurries were mixed with complete media containing 0.03% DNAase (2650 Kunitz units/mL) and 0.14% collagenase I (both enzymes obtained from Sigma Chemical Co., St. Louis, Mo.), placed into 50 ml Erlenmeyer flasks with stirring, and incubated for 90 min at 37° C. under a humidified 5% CO₂ atmosphere. After enzymatic dispersion into a near single cell suspension, tumor cells were filtered through nylon mesh, and washed in complete media. A portion of the cell suspension was used for cytospin slide preparation and stained with Wright-Giemsa for examination by a medical pathologist in parallel with Hematoxylin-Eosin stained tissue sections to confirm the diagnosis and to determine the tumor cell count and viability. Tumor cells were then suspended in soft agarose (0.13%) and plated at 20,000-50,000 cells per well onto an agarose underlayer (0.4%) in 24-well plates. Tumor cells were incubated under standard culture conditions for 4 days in the presence or absence of a cytotoxic concentration of paclitaxel (2.45 μM) or docetaxol (2.4 μM). Cells were pulsed with tritiated thymidine (New Life Science Products, Boston, Mass.) at 5 μCi per well for the last 48 hours of the culture period. After labeling, cell culture plates were heated to 96° C. to liquify the agarose, and the cells are harvested with a micro-harvester (Brandel, Gaithersburg, Md.) onto glass fiber filters. The radioactivity trapped on the filters was counted with an LS-6500 scintillation Counter (Beckman, Fullerton, Calif.). Untreated cells served as a negative control. In the positive (background) control group, cells were treated with a supratoxic dose of Cisplatin (33 μM), which causes 100% cell death. Detectable radioactivity for this group was considered non-specific background related to debris trapping of tritiated thymidine on the filter. After subtracting background control values, percent control inhibition (PCI) of proliferation was determined by comparing thymidine incorporation by the treatment group with incorporation by the negative control group: PCI=100% ×[1−(CPM treatment group, CPM control group)]. Determinations of docetaxol effects on tumor proliferation were performed in duplicate or triplicate. Breast cancer tumor cell lines tested in the EDR assay were handled in a fashion comparable to solid tumors and plated at 1,000-5000 cells per well. Cell lines were harvested with trypsin and washed twice in phosphate buffered saline (PBS) prior to their addition to the culture plates.

### Immunoseparation of Endothelial Cells

Immunoseparation of human breast cancer tumor-derived vascular endothelial cells (VECS) were performed according to the teachings of co-owned and co-pending U.S. Ser. No. 10/144,142 filed May 10, 2002, the disclosure of which is incorporated by reference herein.

Immunomagnetic isolation using magnetic beads provides a simple and reliable method for positive or negative isolation and enrichment of VEC that are present at low concentrations (<1%) in mixed cell populations. Dynabeads (Dynal, Oslo, Norway) are highly uniform, supermagnetic polystyrene spheres coated with mono- or polyclonal antibodies. Antibodies can be conjugated with immunobeads either directly via covalent bonds or indirectly, via a DNA linker, allowing for the release of isolated cells from the beads upon capture using DNase-releasing buffer. The released populations of endothelial cells can be subsequently verified for purity, cultured in different growth environments as described above, and re-analyzed using mAbs against VEC differentiation markers and/or functional test as described above. As an example of negative selection, Dynabeads conjugated with mouse mAb against human CD45 were used for CD31- and/or CD105-positive subsets of hematopoietic cells contaminating tumor cell specimens (macrophages, granulocytes, lymphocytes).

The CELLection Pan Mouse IgG Kit (Dynal) was used in these studies. Cells were stained with unlabeled anti-CD105 mAb (Becton Dickinson), washed and analyzed by flow cytometry to verify the CD105 positivity of HUVEC and CD105 negativity of other breast cancer tumor samples, as described below. The mixture was separated under sterile conditions using Dynabeads conjugated with polyclonal anti-mouse IgG antibodies, the unbound (CD105-negative) cells and the bound (CD105-positive) cells were separately collected. The bound cells were released from the beads using the DNase buffer. Aliquots from both cell suspensions were then analyzed by flow cytometry for the expression of CD105. 90% of positively selected cells were CD105-positive, while 99% of the negatively selected population was CD105-negative. Both populations were plated on Becton Dickinson BIOCOAT flasks covered with collagen I 48 hours after plating and analyzed by flow cytometry for CD105 expression and by immunofluorescence for Dil-ac-LDL uptake. No CD105 expression and no Dil-ac-DLD uptake was found in negatively selected cells, while 79% of positively selected cells expressed CD105 on the membrane and actively took up Dil-ac-LDL. No CD45-expressing cells were found in the positively selected population by flow cytometry.

Using a complementary approach, a FACSVantage Turbosort flow cytometer was used to achieve highly efficient VEC immunoseparation. Human breast cancer tumor cells were analyzed before sorting for CD105 expression, and the two cell populations (CD105+ and CD105−) were identified and gated individually. A sterile flow sort was then performed based on CD105 staining. CD105-positive and CD105-negative cells were collected in two separate tubes and re-analyzed for CD105 expression. In these experiments, the purity in CD105+ and CD105− sorted populations were 99.84% and 99.91%, respectively. Total RNA preparations were then isolated from these cells and analyzed using human gene arrays, as described below.

While flow cytometry-based sorting was highly efficient and reproducibly isolated purified (>99%) VEC, this technique can be time-consuming, particularly when sorting rare events. Because the percentage of VEC does not exceed 1% in the vast majority of clinical specimens, tumor samples are enriched by the use of immunomagnetic separation, subsequently followed by flow sorting. In this combined approach, tumor cell suspensions containing <1% of VEC is first enriched by one or two orders of magnitude using immunobeads and then subjected to highly efficient flow cytometry sorting procedures that yield >99% pure cell populations. Pure VEC, as well as sorted cells that are negative for endothelial differentiation markers, are further analyzed by morphological, functional and molecular biology techniques.

### Tissue Culture and Cell Expansion

All breast cancer tumor cell lines, such as MCF-7 (obtained from the American Type Culture Collection (ATCC), Manassas, Va.) were maintained in RPMI 1640 (GibcoBRL, Grand Island, N.Y.) supplemented with 10% fetal calf serum (FCS, Gemini Bioproducts, Inc., Calabasas, Calif.), 100 IU/ml penicillin, 100 mg/ml streptomycin, and 2 mM L-glutamine (all purchased from Irvine Scientific, Irvine, Calif.) (this mixture is termed “complete medium” herein). Cells were harvested with 0.25% trypsin (GibcoBRL) after washing twice with phosphate-buffered saline (PBS, Irvine Scientific), then washed with complete medium, counted and checked for viability using trypan blue or propidium iodide (PI)-based flow cytometry, and processed for flow cytometry analysis or sorting.

In order to provide sorted tumor cells with optimal growth conditions, Becton Dickinson BIOCOAT microenvironment cell culture system was used. Tissue culture flasks (T-25, T-75, T-175, and T-225, Becton Dickinson, San Jose, Calif.) coated with rat-tail collagen I as a substrate for adhesion and growth of neoplastic cells were used in all experiments to expand sorted populations for gene array and cell sorting studies.

To approximate in vitro conditions for the growth neoplastic cells to the in vivo growth environment, Ultra Low Attachment 24-well plates (Costar, N.Y.) comprised of a covalently bound hydrogel layer that is hydrophobic and neutrally charged. This hydrogel surface inhibited non-specific immobilization of anchorage-dependent neoplastic cells via hydrophobic and ionic interactions and created an in vitro environment for culturing sorted and expanded neoplastic cells in organoid cultures. In titration experiments, the SKBR3 cell line (human breast cancer cell line, obtained from the ATCC) was plated in 24-well cytophobic plates at 500,000 cells per well, in duplicate, and treated with docetaxol at a concentration of 2.4 uM in complete medium for differing lengths of time ranging from 24 to 72 hours.

### Flow Cytometry Analysis and Cell Sorting

Samples of viable neoplastic cells were immediately analyzed on Becton Dickinson FACSort or FACSVantage flow cytometers equipped with a Coherent Enterprise laser tuned to 488 nm. Forward scatter, side scatter, FL-1 (FITC, fluorescein isothiocyanate), FL-2 (PE, phycoerythrin), and FL-3 (PI, propidium iodide) parameter data were collected in list mode. 10,000 events per sample were collected and consequently analyzed using the Becton Dickinson CellQuest flow cytometry acquisition software. In all samples, PI was added to exclude dead cells. Data shown are PI negative (viable) cells.

Flow sorting was performed on the Becton Dickinson FACSVantage instrument using the following parameters. In surface tumor-specific marker-based flow sorting, neoplastic cells numbers and viability were determined using the FACScan. Cells were the washed in 45 ml of serum-free RPMI and centrifuged at 1572×g (4° C., 5 min). An aliquot (0.5−1×10⁶ cells) was labeled by the isotype control preparation (mouse IgGI from Sigma, at a final concentration of 2 μg/mL) at 4° C. for 30 min. The remaining cells were labeled under the same conditions with the 9GG.10 anti-human HER2 monoclonal antibody (mAb) at 2 μg/mL (Neomarkers, Fremont, Calif.) or the 111.6 anti-human EGF-R mAB (Neomarkers). Cells were washed twice by ice-cold serum-free RPMI and centrifuged at 1572×g (4° C., 5 min). Washed cells were then labeled on ice with phycoerythrin (PE)-labeled anti-mouse IgGI for 30 min, washed again in ice-cold PBS +1% FCS (1572×g, 4° C., 5 min), re-suspended in cold PBS+1% FBS supplemented with PI (1:g/mL), and sorted on the FACSVantage. Sorted breast cancer cells were collected in a 5 mL plastic tube containing 2 mL of a 50/50 mixture of serum-free RPMI and FCS. Cell counts were recorded from the FACSVantage. After one wash with cold PBS (centrifugation at 1572×g, 4° C., 5 min), cell numbers and viabilities were determined using the FACScan. Cell pellets were used for RNA extraction if at least 2×10⁶ viable sorted HER2⁺ or EGFR⁺ cells were recovered. An additional aliquot of 5×10⁵ cells were expanded for further analysis. If less than 2.5×10⁶ viable sorted cells were recovered, all sorted cells were cultured in vitro for further analysis. Purity (defined as the percentage of neoplastic cells in the sample) and viability of the sorted populations were determined using the FACSVantage.

Flow sorting based on Annexin V binding was performed using the same protocol and FITC-labeled Annexin V (PharMingen, San Diego, Calif.), with the following modifications. The following controls were used to set up compensation and quadrants: (1) unstained cells (autofluorescence control), (2) cells stained with Annexin V-FITC only (no PI), and (3) cells stained with PI only (no Annexin V-FITC). Washed neoplastic cells were mixed with Annexin V-FITC (5:L of the probe per 1×10⁵ cells) and/or PI (10:L of 50:g/mL stock solution per 1×10⁵ cells), gently vortexed and incubated at room temperature (20-25° C.) in the dark for 15 min. Annexin V-labeled cells were then re-suspended in 1× binding buffer provided by PharMingen and sorted on the FACSVantage, as recommended by the manufacturer. The following cell populations are separated: Annexin V+/PI− (sensitive cells) and Annexin V−/PI− (resistant cells). Sorted cells were collected in a FACS tubes, and purity and viability of the sorted populations were determined using the FACSVantage as described above.

### Human Breast Carcinoma Tumor Sample

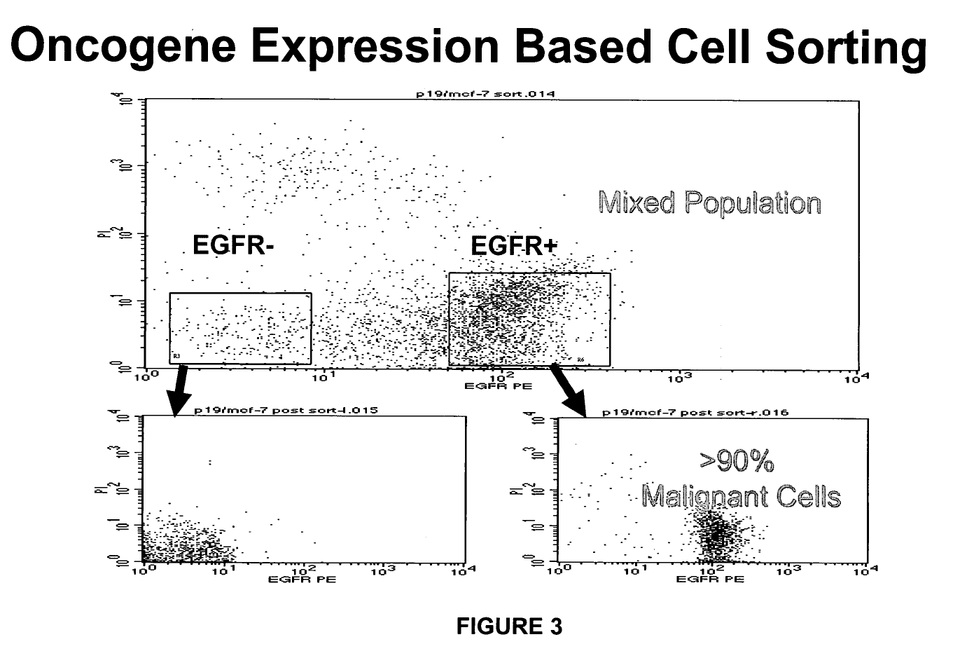

Results of cell sorting experiments as described above with disaggregated cells from a human breast carcinoma tumor sample are shown in FIGS. 3A through 3C. Viable neoplastic human breast carcinoma cells were separated as described above from the disaggregated tumor sample and treated with cytotoxic drugs. These cells were then sorted after treatment with the apoptosis-discriminating agent Annexin V to separate living, drug resistant cells from apoptotic, drug sensitive cells. FACS analysis after propidium iodide staining is shown in FIG. 3A, where about 60% of the cells were viable. FIGS. 3B and 3C show separation of the population into apoptotic, drug-sensitive neoplastic cells (<10%) and living, drug-resistant neoplastic cells (>90%).

A total of 10 taxane-resistant and 7 taxane-sensitive human breast cancer tumor samples were identified using these assays, the cells of which were used in gene array analyses of differential gene expression disclosed in Example 2.

### EXAMPLE 2

### Gene Array Analyses

Neoplastic cells prepared from freshly resected human breast cancer tumors by FACS sorting as described in Example 1, breast cancer tumor-derived vascular endothelial cells, and human breast cancer cell lines were used to make mRNA for performing gene array hybridization analyses. Differential gene expression was analyzed between these different cell types sorted into taxane-sensitive and -resistant populations.

RNA Isolation

Cells were collected by gentle centrifugation (about 1500×g) to preserve their integrity. After isolating the pelleted cells from the supernatant fluid, the cells were lysed in TRIzol® Reagent (Life Technologies™, Rockville, Md.) by repetitive pipetting, using about 1 mL of Trizol reagent per 1−10×10⁶ cells. The lysed cell sample was then incubated for 5 minutes at room temperature to permit the complete dissociation of nucleoprotein complexes. To this mixture was added about 0.2 mL chloroform per 1 mL of Trizol Reagent and the tube shaken vigorously and then incubated at room temperature for 2 minutes. The organic and aqueous phases were separated by centrifugation at about 12,000×g for 15 minutes at 5° C. The aqueous phase was carefully collected and transferred to a fresh tube, and the RNA precipitated by mixing with mixing with 0.5 mL of isopropyl alcohol. The samples were then incubated at room temperature for 10 minutes and centrifuged at 12,000×g for 10 minutes at 5° C. The supernatant was carefully removed from the RNA pellet, which was then washed once with 1 mL of 75% ethanol. The ethanol was removed and the RNA pellet air-dried for 10 minutes. Finally, the RNA pellet was dissolved in RNase-free water by incubating for 10 minutes at 55° C.

The yield and purity of total RNA was determined spectrophotometrically. The integrity of the purified RNA was determined by agarose gel electrophoresis using conventional methods.

MicroArray Assay

cDNA Probe Preparation

cDNA was prepared from cellular RNA as follows. Total RNA (5 to 15 μg) was used to generate double-stranded cDNA by reverse transcription using a cDNA synthesis kit (Superscript Choice System, Life Technologies, Inc., Rockville, Md.) that uses an oligo(dT)₂₄ primer containing a T7 RNA polymerase promoter 3′ to the poly T (Geneset, La Jolla, Calif.), followed by second-strand synthesis. Labeled cRNA was prepared from the double-stranded cDNA by in vitro transcription by T7 RNA polymerase in the presence of biotin-11-CTP and biotin-16-UTP (Enzo, Farmington, N.Y.). The labeled cRNA was purified over RNeasy columns (Qiagen, Valencia, Calif.). Fifteen μg of cRNA was fragmented at 94° C. for 35 minutes in 40 mmol/L of Tris-acetate, pH 8.1, 100 mmol/L of potassium acetate, and 30 mmol/L of magnesium acetate. The cRNA was then used to prepare 300 μL of hybridization cocktail (100 mmol/L MES, 1 mol/L NaCl, 20 mmol/L ethylenediaminetetraacetic acid, 0.01% Tween 20) containing 0.1 mg/mL of herring sperm DNA (Promega, Madison, Wis.) and 500 μg/mL of acetylated bovine serum albumin (Life Technologies, Inc.). Before hybridization, the cocktails were heated to 94° C. for 5 minutes, equilibrated at 45° C. for 5 minutes, and then clarified by centrifugation (16,000×g) at room temperature for 5 minutes.

Hybridization

Microarrays (U133A) were obtained from Affymetrix (Santa Clara, Calif.) and used according to the manufacturer's instructions. The U133A gene chip contained over 22,000 sequences derived from known genes and expressed sequence tags (ESTs).

Aliquots of the hybridization cocktail described above containing 15 μg of fragmented cRNA were hybridized to U133A arrays at 45° C. for 16 hours in a rotisserie oven at 60 rpm. After hybridization, the gene chips are automatically washed and stained with streptavidin-phycoerythrin using a fluidics station as follows: the arrays are washed using nonstringent buffer (6×SSPE) at 25° C., followed by stringent buffer (100 mmol/L MES, pH 6.7, 0.1 mol/L NaCl, 0.01% Tween 20) at 50° C. The arrays were stained with streptavidin-phycoerythrin (Molecular Probes, Eugene, Oreg.), washed with 6× sodium chloride, sodium phosphate, EDTA (SSPE buffer), incubated with biotinylated anti-streptavidin IgG, stained again with streptavidin-phycoerythrin, and washed again with 6× SSPE. The arrays were scanned using the GeneArray scanner (Affymetrix). Image analysis was performed with GeneChip software (Affymetrix).

Expression levels of 3′ and 5′ signals for both GAPDH and b-actin housekeeping genes were evaluated for quality control of sample preparation. Data were normalized to an internally consistent set of 100 probe sets as determined by Affymetrix® and scaled to a value of 500. This approach allows a unified data set that can be compared across all other samples.

The arrays were scanned at 3-mm resolution using the Genechip System confocal scanner made for Affymetrix by Agilent (Agilent G2565AA DNA microarray scanner). Microarray Suite 5.1 software from Affymetrix was used to determine the relative abundance of each gene, based on the average difference of intensities. Data analysis began with scanning, which collected data for each feature, containing an identical sequence set of 25-mers in an 18 μm area. Each feature was scanned 6 times to collect a 6×6 set of pixels covering the 18 μm area. Only the inner set of 4×4 pixels were read as the probe pixel set to avoid collection of signal bleed from adjacent elements. The chip was segmented into 16 zones, and a background correction was applied by subtracting the lowest 2% of signal values calculated for these zones adjusted by a distance weighting such that the local background within a zone contributed more heavily to the 2% calculation than do more distant zones. Thus, each zone had its own unique 2% background correction value. After background correction, the 16 signal values for each reading set were arranged into a normal distribution, and the signal value that fell at the 75^{th} percentile was selected as the final feature signal. These data were collected in a file. The raw output of the scanned image was visually inspected prior to further data analysis to assure that no fractures in the chip surface occurred during processing, and that the signal strength was uniform on the chip.

Raw data obtained from hybridization experiments was analyzed using bioinformatics tools to identify signals associated with differential gene expression. Basic statistical methods such as t-test with a p value <0.05 and fold change >1.5 were performed by Affymetrix® Data Mining Tool 3.0. Differential gene expression results were combined in a Venn Diagram approach to find common sets of genes found from multiple methods. Hierarchical clustering was also used, where a distance matrix was first calculated, containing the distances between every pair of specimens in the data set. A tree was then built by merging the two closest specimens until all of the specimens were contained in the tree. Hierarchical Cluster Analysis was performed using either Cluster and TreeView Software (http://rana.lbl.gov/EisenSoftware.htm) or GeneSpring (Silicon Genetics).

Hybridization experiments as described herein were performed using cRNA prepared from FACS sorted populations of drug sensitive and drug resistant populations of sorted (>90%) human breast cancer tumor cells, human breast cancer tumor-derived vascular endothelial cells, and human breast carcinoma cells as described in Example 1. Results were obtained for differential gene expression associated with intrinsic drug resistance, i.e. differences in gene expression from drug-sensitive and drug-resistant cells detected without prior taxane treatment, and for induced drug sensitivity or resistance, i.e. after cell growth in the presence of 0.1 mM docetaxel. Results of differential gene expression assays showing an association with intrinsic drug sensitivity and resistance are shown in Table I, and results of differential gene expression associated with intrinsic drug sensitivity and resistance are shown in Table II. In both types of resistance, differential gene expression was detected for both sensitive and resistant cells.

For intrinsic taxane drug response, analysis using a p value <0.05 and fold change >2.5 revealed 26 differentially expressed genes (Table I).

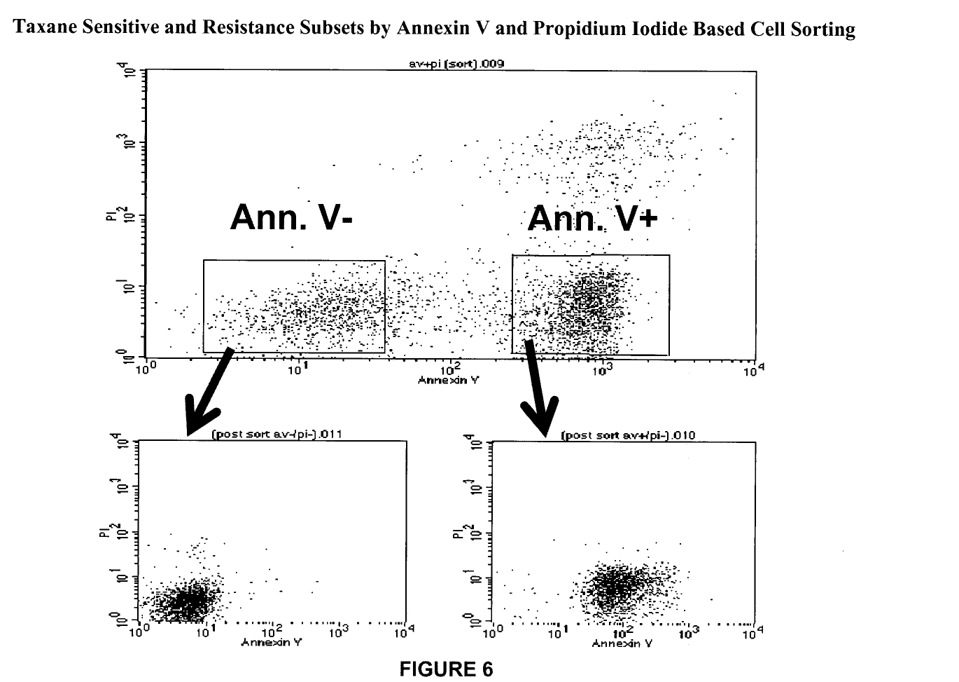

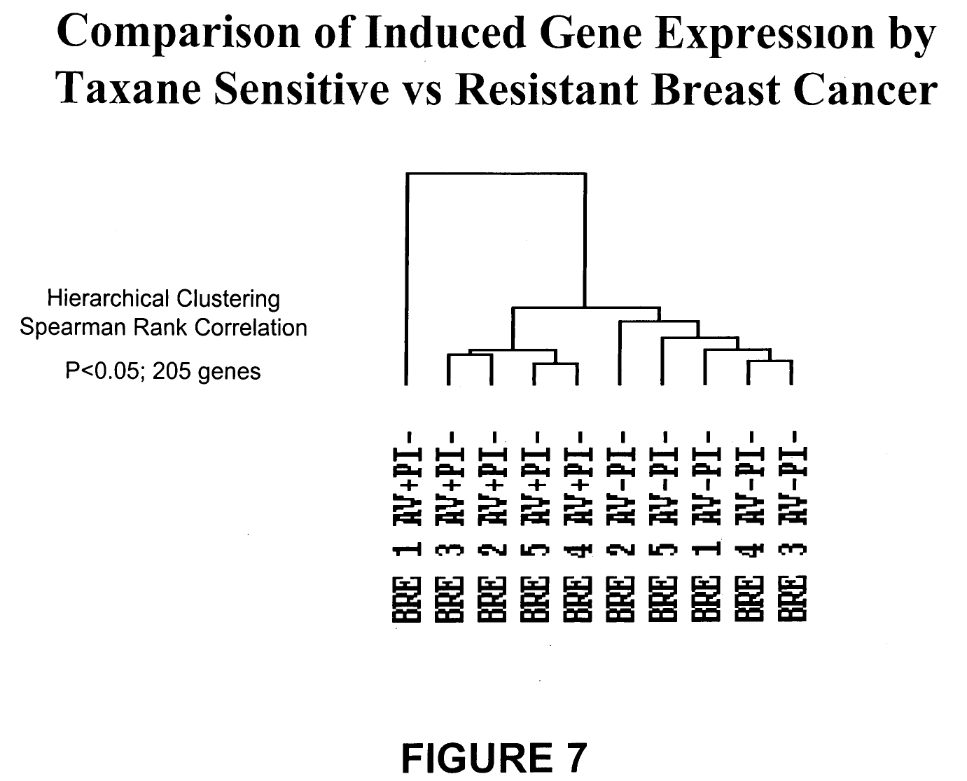

Five breast cancer specimens were treated with docetaxel (0.1:M) and sorted into sensitive and resistant subsets using Annexin V as a marker of apoptosis (FIG. 6). Microarray analyses were performed on breast cancer specimens with induced taxane drug response based on binding of Annexin V. Analysis using a p value <0.05 and fold change >1.5 revealed 8 differentially expressed genes (Table II). Hierarchical cluster analysis using a spearman rank correlation accurately separated the sensitive and resistant subsets using 205 genes based on a p value<0.05 (FIG. 7).

Alternatively, GeneFilters® (Research Genetics) membranes were used for microarray analyses. In these embodiments, the microarray filters were washed for at least 5 minutes with gentile agitation in a boiling (95-100° C.) solution of 0.5% SDS to remove manufacturing residuals and are then prehybridized in 5 mL of MicroHyb hybridization solution (Research Genetics) with 5.0 μg Cot-1 DNA, used as a blocker for repeat sequences that decreases the background of hybridizations, (Human Cot-1 DNA, Life Technologies) and 5.0 μg poly dA (1 μg/uL, Research Genetics) in a roller oven (Hybaid, Midwest Scientific St. Louis, Mo.) at 42° C. for 4 to 6 hours. For each labeling, total RNA corresponding to 1 μg was reverse transcribed in the presence of 10 μL of ³³P dCTP (10 mCi/mL with a specific activity of 3000 Ci/mmol, ICN Radiochemicals, Costa Mesa, Calif.), 2.0 μL oligo dT (1 μg/μL of 10-20 mer mixture, Research Genetics), and 300 units of Reverse Transcriptase (Superscript II, Life Technologies). The samples were incubated for 90 minutes at 37° C., and cDNA probes purified by passaging through a Bio-Spin 6 chromatography column (Bio-Rad, Hercules, Calif.) to remove any unincorporated nucleotides. The cpm counts of the probes were measured to confirm successfull labeling. The GeneFilters® membrane were hybridized with the probes overnight (12-18 hours) at 42° C. in a hybridization roller oven at 8-10 rpm. The membranes were then washed twice with 30 mL of 2×SSC containing 1% SDS at 50° C. for 20 minutes and once with 30 mL of 0.5× SSC containing 1% SDS at 55° C. for 15 minutes in hybridization oven at 12-15 rpm. After washing, the GeneFilter® membrane was placed on a filter paper moistened with deionized water and wrapped with a plastic film.

GeneFilters® membranes were then exposed overnight to a Packard phoshor imaging screen and scanned at 600 dpi resolution in a Cyclone phoshor imaging system (Packard Instrument Co., Meriden, Conn.). Resulting images in the tiff format were directly imported into the image analysis software Pathways® 3 (Research Genetics). The software used control spots present throughout the filter to align the images and performs auto-centering of the spots.

ResGen™ GeneFilters® were performed on purified breast cancer cells, purified tumor-derived endothelial cells, and a MCF7 breast cancer cell line. Taxane sensitive gene expression profiles were compared to taxane resistant gene expression profiles and 16 genes were found to be differentially expressed.

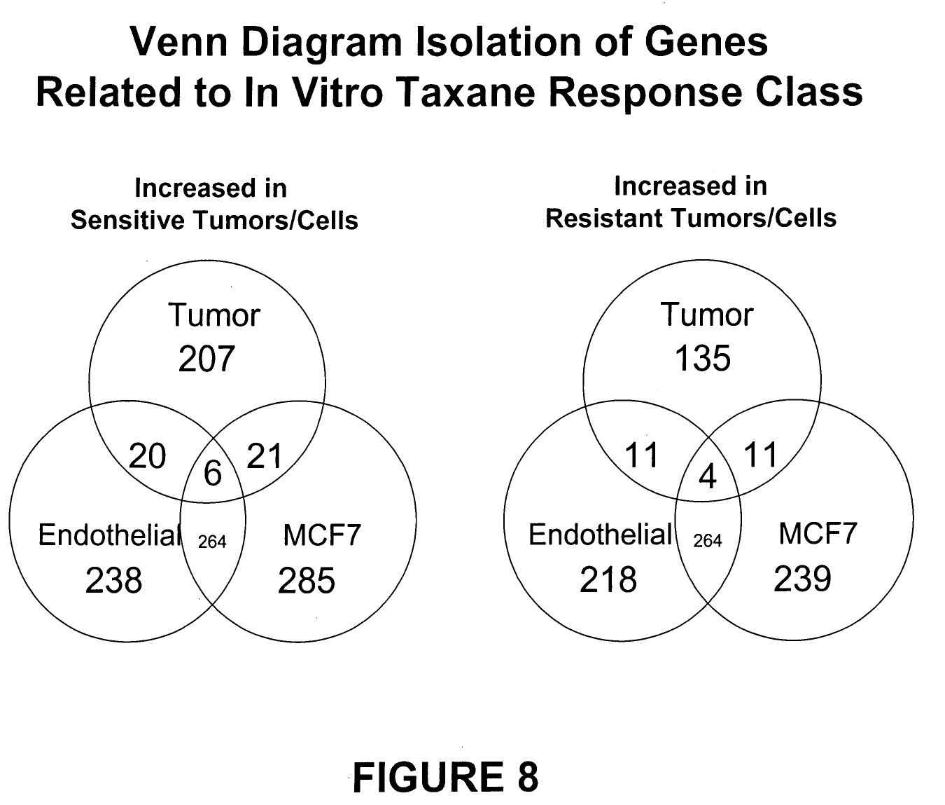

Differential gene expression of purified breast cancer cells, purified tumor-derived endothelial cells, and a MCF7 breast cancer cell line were combined in a Venn Diagram (FIG. 8). Six genes were found to be consistently increased in taxane sensitive tumors/cells and 4 genes were found to be consistently increased in taxane resistant tumors/cells (Table III).

TABLE I
Comparison of Intrinsic Gene Expression by Taxane Sensitive vs Resistant
Breast Cancer

Increased in Sensitive Samples
Islet cell autoantigen 1, transcript variant 2
Ribonucleotide reductase M2 polypeptide
Rho GDP dissociation inhibitor (GDI) beta
CDC28 protein kinase 1
PTPL1-associated RhoGAP 1
MAD2 (mitotic arrest deficient, yeast, homolog)-like 1
sodium channel, nonvoltage-gated 1 alpha
EST hypothetical protein MGC3077
EST hypothetical protein FLJ10430
EST hypothetical protein FLJ23468
Increased in Resistant Samples
Ser-Thr protein kinase related to the myotonic dystrophy protein kinase
SMC family member, chromosome-associated protein E
Pleckstrin homology-like domain, family A, member 1
WEE1 gene for protein kinase and partial ZNF143 gene for zinc finger
transcription factor
GABA-A receptor-associated protein
V-ETS avian erythroblastosis virus E26 oncogene homolog 2
Kruppel-like factor 4 (gut)
EST from clone DKFZp586F071
Secretory granule, neuroendocrine protein 1

TABLE II
Comparison of Induced Gene Expression by Taxane Sensitive vs Resistant
Breast Cancer

Increased in Sensitive (AV + PI-)
H2A histone family, member Y
Increased in Resistant (AV − PI-)
Interferon-induced protein with tetratricopeptide repeats 1
Basic kruppel like factor
Two-pore channel 1
Heterogeneous nuclear ribonucleoprotein A0
Chimerin 2
EST AA181053
ADP-ribosylation factor related protein 1

P<0.05; Fold Change≧1.5; 8 genes

TABLE III
Overlapping Gene Sets For Taxane Sensitive VS. Resistant Solid Tumors,
MCF7 Breast Cancer Cells and Vascular Endothelial Cells

Increased in Sensitive Specimens
Sjogren syndrome antigen B (autoantigen La)
Capping protein alpha
Adenine nucleotide translocator 3 (liver)
AU-rich element RNA-binding protein AUF1
Phosphatidyl serine synthase I
Integrin, alpha L, lymphocyte function-associated antigen 1
Branched chain keto acid dehydrogenase E1, alpha polypeptide
Annexin XI (56 kD autoantigen)
Von Hippel-Lindau syndrome
Increased in Resistant Specimens
Myosin phosphatase target subunit 1 (MYPT1)
Albumin D-box binding protein
Complement component 7
Plasminogen activator, urokinase receptor
ATPase, DNA-binding protein (HIP116)
Zinc finger protein (ZNF198)
Tropomodulin

It should be understood that the foregoing disclosure emphasizes certain specific embodiments of the invention and that all modifications or alternatives equivalent thereto are within the spirit and scope of the invention as set forth in the appended claims.

We claim:

1. A method for identifying genes having an expression pattern in tumor cells that is modulated when the cells are contacted with a chemotherapeutic agent comprising the steps of:
a) separating living neoplastic cells from dead cells, vascular endothelial cells and living stromal cells in a mixed population of cells from a tumor sample, by
i) contacting, the mixed population of cells with a vital stain or fluorescent dye;
ii) contacting the mixed population of cells with a detectably-labeled immunological reagent that specifically binds to neoplastic cells; and
iii) selecting the cells in the mixed population of step (b) that are not stained with the vital stain and that bind the immunological reagent,
b) contacting the separated, living neoplastic cells with a chemotherapeutic amount of a chemotherapeutic agent;
c) separating apoptotic and non-apoptotic cells of step b) by contacting the cells with a reagent specific for apoptosis and sorting the population of cells in step b) thereby;
d) assaying gene expression in each of the separated populations of apoptotic and non-apoptotic cells; and
e) identifying genes having an expression pattern that is modulated by contacting cells with a chemotherapeutic amount of a chemotherapeutic agent.

2. A method according to claim 1, wherein expression of one or a plurality of genes is increased in cells sensitive to the chemotherapeutic agent.

3. A method according to claim 1, wherein expression of one or a plurality of genes is increased in cells resistant to the chemotherapeutic agent.

4. A gene whose expression is increased according to the method of claim 2, wherein the gene is Sjogren syndrome antigen B (autoantigen La), capping protein alpha, adenine nucleotide translocator 3 (liver), AU-rich element RNA-binding protein AUF1, phosphatidylserine synthase I, integrin, alpha L, lymphocyte function-associated antigen 1, branched chain keto acid dehydrogenase E1, alpha polypeptide, annexin XI (56 kD autoantigen), or Von Hippel-Lindau syndrome.

5. A gene whose expression is increased according to the method of claim 3, wherein the gene is myosin phosphatase target subunit 1 (MYPT1), albumin D-box binding protein, complement component 7, plasminogen activator, urokinase receptor, ATPase, DNA binding protein (HIP 116), zinc finger protein (ZNF198) or tropomodulin.

6. A method for identifying one or a plurality of genes having a pattern of expression that is different in a tumor cell sensitive to a chemotherapeutic drug than the expression pattern in a tumor cell resistant to the chemotherapeutic drug, the method comprising the steps of:
a) performing an extreme drug resistance (EDR) assay on a mixed population of cells from a tumor sample;
b) separating living tumor cells from dead cells, vascular endothelial cells and living stromal cells in a mixed population of cells from a tumor sample, by
i) contacting the mixed population of cells with a vital stain or fluorescent dye;
ii) contacting the mixed population of cells with a detectably-labeled immunological reagent that specifically binds to neoplastic cells; and
iii) selecting the cells in the mixed population that are not stained with the vital stain and that bind the immunological reagent,
c) assaying gene expression in each of the separated populations of drug sensitive and drug resistant cells; and
d) identifying genes having an expression pattern that is different in the drug resistant cells than in the drug sensitive cells.

7. A method according to claim 6, wherein expression of one or a plurality of genes is increased in cells resistant to the chemotherapeutic agent.

8. A method according to claim 6, wherein expression of one or a plurality of genes is increased in cells sensitive to the chemotherapeutic agent.

9. A method according to claim 1, wherein expression of one or a plurality of genes is decreased in cells resistant to the chemotherapeutic agent.

10. A method according to claim 1, wherein expression of one or a plurality of genes is decreased in cells sensitive to the chemotherapeutic agent.

11. A gene whose expression is increased according to the method of claim 8, wherein the gene is Sjogren syndrome antigen B (autoantigen La), capping protein alpha, adenine nucleotide translocator 3 (liver), AU-rich element RNA-binding protein AUF1, phosphatidylserine synthase I, integrin, alpha L, lymphocyte function-associated antigen 1, branched chain keto acid dehydrogenase E1, alpha polypeptide, annexin XI (56 kD autoantigen), or Von Hippel-Lindau syndrome.

12. A gene whose expression is increased according to the method of claim 7, wherein the gene is myosin phosphatase target subunit 1 (MYPT1), albumin D-box binding protein, complement component 7, plasminogen activator, urokinase receptor, ATPase, DNA binding protein (HIP 116), zinc finger protein (ZNF198) or tropomodulin.

13. The method of claim 1, wherein the cell is a breast cancer tumor cell.

14. The method of claim 6, wherein the cell is a breast cancer tumor cell.

15. The method of claim 1, wherein the chemotherapeutic drug is a taxane.

16. The method of claim 6, wherein the chemotherapeutic drug is a taxane.

17. The method of claim 15, wherein the chemotherapeutic drug is a taxane, docetaxel or paclitaxel.

18. A pattern of gene differential gene expression, comprising increased expression of one or a plurality of genes that are myosin phosphatase target subunit 1 (MYPT1), albumin D-box binding protein, complement component 7, plasminogen activator, urokinase receptor, ATPase, DNA binding protein (HIP 116), zinc finger protein (ZNF198) or tropomodulin, wherein increased expression of said one or plurality of genes in a tumor cell compared with a non-tumor cell identifies said tumor cell to be a cell resistant to chemotherapeutic drugs that are taxanes.

19. A pattern of gene differential gene expression, comprising increased expression of one or a plurality of genes that are Sjogren syndrome antigen B (autoantigen La), capping protein alpha, adenine nucleotide translocator 3 (liver), AU-rich element RNA-binding protein AUF1, phosphatidylserine synthase I, integrin, alpha L, lymphocyte function-associated antigen 1, branched chain keto acid dehydrogenase E1, alpha polypeptide, annexin XI (56 kD autoantigen), or Von Hippel-Lindau syndrome wherein increased expression of said one or plurality of genes in a tumor cell compared with a non-tumor cell identifies said tumor cell to be a cell sensitive to chemotherapeutic drugs that are taxanes.

20. A method for identifying a tumor or cells comprising the tumor that are resistant to taxane chemotherapeutic drugs, the method comprising the steps of:
a) determining gene expression levels in a tumor sample or cells comprising the tumor for one or a plurality of genes that are myosin phosphatase target subunit 1 (MYPT1), albumin D-box binding protein, complement component 7, plasminogen activator, urokinase receptor, ATPase, DNA binding protein (HIP 116), zinc finger protein (ZNF198) or tropomodulin;
b) comparing gene expression levels of the one or plurality of genes in step a) with gene expression levels of said one or plurality of genes in a non-tumor sample or cells comprising said sample; and
c) identifying the tumor or cells comprising the tumor to be resistant to taxane chemotherapeutic drugs when the gene expression levels of one or a plurality of said genes is increased in the tumor sample when compared to gene expression levels in the non-tumor sample.

21. The method of claim 20, wherein the tumor sample is a breast cancer tumor sample.

22. A method for identifying a tumor or cells comprising the tumor that are sensitive to taxane chemotherapeutic drugs, the method comprising the steps of:
a) determining gene expression levels in a tumor sample or cells comprising the tumor for one or a plurality of genes that are Sjogren syndrome antigen B (autoantigen La), capping protein alpha, adenine nucleotide translocator 3 (liver), AU-rich element RNA-binding protein AUF1, phosphatidylserine synthase I, integrin, alpha L, lymphocyte function-associated antigen 1, branched chain keto acid dehydrogenase E1, alpha polypeptide, annexin XI (56 kD autoantigen), or Von Hippel-Lindau syndrome;
b) comparing gene expression levels of the one or plurality of genes in step a) with gene expression levels of said one or plurality of genes in a non-tumor sample or cells comprising said sample; and
c) identifying the tumor or cells comprising the tumor to be sensitive to taxane chemotherapeutic drugs when the gene expression levels of one or a plurality of said genes is increased in the tumor sample when compared to gene expression levels in the non-tumor sample.

23. The method of claim 21, wherein the tumor sample is a breast cancer tumor sample.

24. The method of claim 1, wherein the vital stain is propidium iodide.

25. The method of claim 1, wherein the immunological reagent is detectably labeled with a fluorescent label.

26. The method of claim 1, wherein the immunological reagent is a tumor-specific antibody.

27. The method of claim 1, wherein the cells of step (c) are selected by fluorescence-activated cell sorting.

28. The method of claim 1, wherein the tumor sample is a solid tumor sample and the mixed cell population is a disaggregated tumor sample.

29. The method of claim 26, wherein the tumor-specific antibody is immunologically specific for EGFR or HER2.

30. A method for detecting a gene expression profile of living neoplastic cells that are resistant to a taxane cytotoxic compound and distinguishing said profile from the gene expression profile of living neoplastic cells that are sensitive to the taxane cytotoxic compound in a mixed population of cells from a tumor sample, the method comprising the steps of:
a) contacting the mixed population of cells with the taxane cytotoxic compound for a time and at a concentration wherein the neoplastic cells that are sensitive to the taxane cytotoxic compound undergo apoptosis;
b) contacting the mixed population of step (a) with a vital stain or fluorescent dye;
c) contacting the mixed population of cells of step (b) with a discrimination compound that specifically binds to apoptotic cells;
d) contacting the mixed cell population of step (c) with a detectably-labeled immunological reagent that specifically binds to the apoptotic cell discrimination compound; and
e) separating the cells in the mixed population of step (d) that are not stained with the vital stain from the cells that are stained with the vital stain;
f) separating the cells in the mixed population of step (e) that are not stained with the vital stain and that do not bind the immunological reagent from the cells in the mixed population of step (c) that are not stained with the vital stain and that do bind the immunological reagent;
g) isolating cellular RNA from the each of the separated cells selected in step (f);
h) preparing detectably-labeled cDNA from the cellular RNA isolated in step (g);
i) hybridizing each of the cDNA preparations prepared in step (h) to a gene array comprising at least 4000 eukaryotic genes;
j) detecting a pattern of gene expression for hybridization of each of the cDNA preparations prepared from the mRNA isolated from the cells selected in step (f); and
k) comparing the pattern of gene expression detected in step (j) from hybridization of the microarray with cDNA from cells that are not stained with the vital stain and that do not bind the immunological reagent with a pattern of gene expression obtained by hybridizing cDNA prepared from cells that are not stained with the vital stain and that do bind the immunological reagent.

31. The method of claim 30, wherein the vital stain is propidium iodide.

32. The method of claim 30, wherein the discrimination compound is Annexin V.

33. The method of claim 32, wherein the immunological reagent specifically binds to Annexin V and is detectably labeled with a fluorescent label.

34. The method of claim 30, wherein the cells of step (f) are selected by fluorescence-activated cell sorting.

35. The method of claim 30, wherein the taxane cytotoxic compound is paclitaxol, taxol or docetaxol.

36. The method of claim 30, wherein the cDNA is detectably labeled with a fluorescent label.

37. The method of claim 30, wherein the mixed population is contacted with the cytotoxic compound under in vitro cell culture conditions whereby the cells cannot attach to a solid substrate.

38. The method of claim 30, wherein the tumor sample is a solid tumor sample and the mixed cell population is a disaggregated tumor sample.

39. The method of claim 38, wherein the tumor sample is a breast cancer tumor sample.
